(19) United States
(12) Patent Application Publication
(10) Pub. No.: US 2009/0010869 A9
(43) Pub. Date: Jan. 8, 2009

(54) Compositions for Topical Enzymatic Debridement

(75) Inventors: Mark W. Trumbore, Westford, MA (US); Roman V. Rariy, Allston, MA (US); Mark Hirsh, Wellesley, MA (US); Jane C. Hirsh, Wellesley, MA (US); Julie A. Saunders, Pocasset, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Collegium Pharmaceutical, Inc.

(21) Appl. No.: 11/843,158

(22) Filed: Aug. 22, 2007

Related U.S. Application Data
(63) Continuation-in-part of application No. 11/147,567, filed on Jun. 8, 2005.
(60) Provisional application No. 60/642,835, filed on Jan. 11, 2005.
(60) Provisional application No. 60/608,991, filed on Sep. 10, 2004.
(60) Provisional application No. 60/604,900, filed on Aug. 27, 2004.
(60) Provisional application No. 60/580,085, filed on Jun. 16, 2004.

(51) Int. Cl. A61K 31/74; A61K 33/38; A61K 35/00; A61K 38/43; A61P 17/00

(52) U.S. Cl. 424/78.08; 424/195.18; 424/618; 424/94.1

(57) ABSTRACT

Formulations are described for the treatment by enzymatic debridement of wounds and ulcers. The formulations have a clear, transparent composition that allows for easy visualization of the wound, and are non-staining for easy clean up. These formulations can also exhibit increased enzymatic debridement activity, improved post-treatment lubricity and coating occlusivity, and stability. The formulations, optionally containing non-animal source biologics, may be in the form of lotions, aerosols to provide a spray, or a foam. A non-reactive substrate may be used as a composition carrier. A non-aqueous lotion formulation having improved enzymatic activity is provided. The non-aqueous lotion viscosity is adjusted to achieve high enzymatic activity while maintaining the application benefits of high viscosity non-aqueous lotions. The lotion formulation may be delivered in a patch.

### PRIORITY

This application is a continuation-in-part of U.S. Ser. No. 11/147,567 filed in the U.S. Patent and Trademark Office on Jun. 8, 2005, by Mark Trumbore, Roman V. Rariy, Mark Hirsh, Jane Hirsh, and Julie Saunders.

### BACKGROUND OF THE INVENTION

Decubitus ulcers, also called decubital ulcers, varicose ulcers, pressure sores or bedsores, typically involve the sacral area, buttocks or lower limbs, particularly at the sites of bony prominences. They most frequently occur in patients, especially the elderly, who have poor circulation. They are commonly found in bed-ridden individuals; in patients with debilitating neuromuscular deficits due to cerebrovascular accidents, dementia, congestive heart failure, or arteriosclerosis; and in individuals with poor nutrition. They can also arise from prolonged pressure on a skin area in an otherwise healthy person. Controlling the progress of these wounds and encouraging their healing is a major challenge both in nursing homes and in the home environment. The wound may be aggravated by the presence of urinary and/or bowel incontinence.

As described in the Merck Manual, there are four stages of pressure sore or ulcer formation. Stages 1 and 2 are generally reversible by relief of pressure. In stage 3, necrosis reaches the underlying fascia, and in stage 4 there is full thickness destruction of skin, and damage to muscle, bone, or supporting structures occurs. Stage 4 ulcers generally require debridement or more extensive surgery. In favorable cases, debridement can be accomplished by non-surgical methods, such as enzymatic debridement. Even after surgery, non-surgical debridement can be used to complete debridement and accelerate healing. Similar issues of wound care are found in patients suffering from burns and open wounds, and the improvements described herein for care of ulcers can be applied to treatment of burns and other open wounds.

Debridement is the removal of debris and damaged or necrotic tissue from a wound, and when needed is an important step in facilitating wound healing. The debridement of these lesions is necessary to remove dead and dying tissue that is typically a source of microbial infection. Healing does not take place until the necrotic tissue is removed. Total therapy for decubitus ulcers includes debridement. While debridement is generally performed surgically, there are instances in which it can be performed by other routes, in particular by treatment of the site with enzymes. This enzymatic treatment is most commonly done with proteolytic enzymes, although other enzyme types, such as polysaccharide-lysing, could also be used.

Enzymes are typically proteins, and require a physiologically compatible environment to function. The enzyme must be functional at a temperature generally close to body temperature. In addition, the enzyme in most cases must be dissolved in water, requires a certain pH of the solution, and often requires cofactors such as metal ions. However, enzymes are often not stable for prolonged periods in aqueous solution, so ways to stabilize an enzyme intended for use in debridement must be provided.

During and after debridement, meticulous care is required to prevent infection during healing, which is often prolonged. This includes maintaining cleanliness and dryness. Careful attention to detail is required, and proper conditions can be difficult to maintain either in an institution or in an isolated setting, such as home care.

There are several products currently on the market for the treatment by enzymatic debridement of ulcers, burns and wounds. These products contain various proteolytic enzymes, including trypsin, papain and collagenase. The vehicles in current use have significant drawbacks. The Balsam of Peru found in some products contains a brownish resin, which stains clothing and bedding. Even heavy bleaching may not remove the stain, especially on non-cotton fabrics, such as polyesters or blends. Viewing the ulcer site is important for evaluating the progress of healing and the early signs of infection. The brown coloration resulting from some of these products partially obscures the view of the ulcer site as does the opaque vehicle found in other products. Moreover, the currently available products cannot readily be delivered uniformly due to the fact that the products are either a liquid (and therefore drip), or a very viscous ointment.

A high viscosity ointment can be difficult and time consuming to apply owing to its low spreadability and the necessity of squeezing it out of a tube. The high viscosity of these ointment formulations makes it difficult to distribute appropriately without undue pressure on the wound, risking adversely disturbing new epithelial growth, which is essential for granulation of the wound, and also causing pain. Additionally, the high viscosity of the ointment can limit the release of proteolytic enzymes from the vehicle, reducing the therapeutic efficacy of the formulation. The currently available aerosol spray formulations contain propane as a propellant, which is flammable and hazardous. The use of this propellant requires special fire suppression equipment during manufacturing, specific storage conditions, and proper disposal. The aerosol spray also has to be held upright in order to spray effectively, which is not always practical with obese or immobile patients. Finally, none of the products contains either buffers to maintain a pH appropriate for optimal enzyme activity or stabilizers that can be used alone or in combination to maximize the activity and/or stability of the enzymes resulting in improved debridement.

It is therefore an object of the present invention to provide improved formulations for wound debridement.

It is an object of the present invention to provide a non-staining formulation.

It is another object of the present invention to provide a stabilized enzyme formulation.

It is still another object of the present invention to provide a non-n y formulation.

It is yet another object of the present invention to provide a more easily sprayable formulation.

### SUMMARY OF THE INVENTION

Formulations having one of more desirable features including non-staining, non-running, more easily sprayable and stabilized enzyme formulation have been developed by changing the formulation, the manufacturing process and/or the packaging system of the formulation. In one embodiment, the composition comprises an improved formulation for the treatment of ulcers that combines debridement and protective agents. In another embodiment, the formulation comprises, besides debridement agents and castor oil or a similar oil phase, a purified form of Balsam of Peru, which has been treated to be non-staining and clear. Typically, the formulation will contain a surfactant to promote efficient dispersion of the various ingredients in the vehicle and facilitate release of the actives from the vehicle.

The formulation preferably contains a powdered trypsin, either from a bovine or a non-bovine source (e.g., recombinant source), or powdered collagenase, papain, chymotrypsin, subtilisn, chymopapain or bromelain. In one preferred embodiment, collagenase is dispersed in an anhydrous carrier. The formulation may comprise any of a number of different grades, both purified and crude, of collagenase. Most collagenase preparations are mixtures of different types of collagenases, and may contain neutral proteases, which can be an advantage in debridement. Neutral proteases can also be added separately to the composition if desired. Other proteases may also be added to the formulations. The carrier may also contain metal and buffer salts to help establish the appropriate environment for optimal enzymatic activity. In another embodiment, trypsin is used as the primary debridement enzyme and is dispersed in an anhydrous carrier. This carrier may optionally contain divalent metal and buffer salts to help establish the appropriate environment for optimal enzymatic activity. In yet another embodiment, at least one proteolytic enzyme such as papain, bromelain, subtilisin, chymotrypsin or chymopapain is used as the primary debridement enzyme and is dispersed in an anhydrous carrier. This carrier may optionally contain one or more buffer salts, mild reducing agents, and divalent metal salts to help establish the appropriate environment for optimal enzymatic activity. The exact combination of additives will be chosen by one skilled in the art to promote rather than inhibit activity.

In another embodiment, the formulation is supplied in a pressurized aerosol can, with a hydrofluoroalkane (HFA) propellant (also known as a hydrofluorocarbon (HFC) propellant), preferably HFA 134a and/or HFA 227. As is well known in the art, changes in propellants frequently lead to deleterious changes in the properties of a formulation. In this aspect, we surprisingly found that the use of HFA propellants have no deleterious effects on the stability and efficacy of the formulation. In a preferred aspect, the formulation is provided in a can with a valve allowing multi-angle spray application, to allow application in a variety of orientations of the can to facilitate coating of ulcers in a variety of locations.

In another aspect, the formulation comprises an emollient, preferably an oil, wax or other hydrophobic material that is a solid at body temperature, which is suspended or preferably dissolved in the formulation, particularly in the presence of the propellant. The increased emolliency imparted by the use of emollients which are solid at room temperature helps to reduce the itching that typically accompanies the regranulation of wounds. The reduction in itching in turn reduces the incidence of scratching and thereby prevents the destruction of newly granulated tissue, thereby helping to promote efficient tissue growth and wound healing.

In another aspect, the formulation comprises a hydrophilic thickener, preferably a colloidal silica or a starch that exhibits shear thinning behavior, allowing efficient aerosolization by the propellant. The increased viscosity imparted by the use of thickeners helps to reduce the itching that typically accompanies the regranulation of wounds. The reduction in itching in turn reduces the incidence of scratching and thereby prevents the destruction of newly granulated tissue, thereby helping to promote efficient tissue growth and wound healing.

In another aspect, the composition may be formulated as a lotion wherein the emollient, preferably an oil, wax or other hydrophobic material, is a solid at body temperature, and is suspended or preferably dissolved in the formulation to provide the appropriate viscosity to be pumped, poured or squeezed from an appropriate container system. The lotion may be applied to a sterile gauze dressing and then placed upon the wound without any undue pressure to the epithelium. Additionally, depending upon the size and location of the wound, the lotion may be dispensed directly onto the site and be left uncovered or dressed appropriately.

In another aspect, the composition may be formulated as a lotion wherein a second emollient, preferably an oil or other hydrophobic material which is a liquid at body temperature, is added in the formulation to provide a more cosmetically elegant skin feel when the formulation is pumped, poured or squeezed from an appropriate container system.

In another aspect, the composition may be formulated as a lotion wherein a hydrophilic thickener, preferably a colloidal silica or a starch that exhibits shear thinning behavior allowing efficient delivery through a pump dispenser, imparts increased viscosity to help reduce the itching that typically accompanies the regranulation of wounds. The reduction in itching in turn reduces the incidence of scratching and thereby prevents the destruction of newly granulated tissue, thereby helping to promote efficient tissue growth and wound healing.

In another aspect, the formulation comprises a non-aqueous lotion comprising castor oil, a purified form of Balsam of Peru, and a sufficient quantity of hydrogenated vegetable oil to modify the viscosity of the non-aqueous lotion. The formulation preferably also contains a debridement agent approved for these uses, and particularly a composition having, as a debridement agent, powdered trypsin or collagen suspended in the formulation, and preferably also contains a surfactant to promote the efficient release of the debriding agent. The final viscosity of the non-aqueous lotion can be adjusted so that it can be delivered from a pump dispenser or by means of a patch system.

In yet another aspect, the formulation comprises a non-aqueous lotion comprising castor oil, a purified form of Balsam of Peru, and a sufficient quantity of colloidal silica to modify the viscosity of the non-aqueous lotion. The formulation preferably also contains a debridement agent approved for these uses, and particularly a composition having, as a debridement agent, powdered trypsin or collagenase suspended in the formulation, and preferably also contains a surfactant to promote the efficient release of the debriding agent. The final viscosity of the non-aqueous lotion can be adjusted so that it can be delivered from an aerosol spray, a pump dispenser or by means of a patch system.

In another aspect, the composition comprises the addition to the formulation of finely powdered excipients that will dissolve in exuded bodily fluids after the formulation is applied to the skin. These include buffers to establish on the surface of the wound the pH conditions most favorable for the enzymatic activity of the debriding enzyme. They may also include divalent metal salts, such as salts of calcium, magnesium and zinc to stabilize the enzyme. In addition, they may also include mild reducing agents such as cysteine. Preferably, the buffer and/or divalent salts and reducing agents are provided as powders, distinct from the powdered enzymes (such as trypsin, collagenase, etc.), so that interaction occurs on the skin and not during storage. Water-soluble polyols may also be used as stabilizers, including glycerol, PEG, sugars and sugar alcohols, and polysaccharides.

In an alternative aspect, the buffers, divalent salts and reducing agents can be provided as an aqueous solution in a separate spray can, or in a hand-pumped sprayer, as part of a kit. The solution, which can be a buffered isotonic solution, can be used to rinse the site before applying the debridement formulation. Another embodiment is a kit containing a first spray can including the improved formulation, a second can comprising a spray anesthetic, and an optional third can or hand pump comprising a rinsing solution.

The compositions have zero shear viscosities greater than about 15,000 cP, more preferably greater than about 25,000 cP, and most preferably greater than about 35,000 cP; and also less than about 700,000 cP, more preferably less than about 550,000 cP and still more preferably less than about 450,000 cP when measured at room temperature. The critical stress values of the compositions are preferably greater than about 25 dynes/cm², more preferably greater than about 75 dynes/cm², and most preferably greater than about 100 dynes/cm², and also preferably lower than about 4,000 dynes/cm², more preferably lower than about 3,000 dynes/cm², and still more preferably lower than about 2,500 dynes/cm², and still more preferably less than 1,500 dynes/cm².

### BRIEF DESCRIPTION OF THE FIGURES

FIG. 1 is a graph showing the average clearance scores as a function of time for three test articles, experimental Formulation 1, experimental Formulation 2 and a commercial trypsin ointment.

FIG. 2 is a graph of zero shear viscosity versus rate of clearance for experimental formulations 4, 5 and 6.

FIG. 3 is a bar graph of the rate of clearance for experimental formulations 4, 5, 6, 7 and 8 showing the effect of metal and buffer salts on enzymatic activity.

### DETAILED DESCRIPTION OF THE INVENTION

### DEFINITIONS

“Ulcer” is used herein to broadly refer to certain skin lesions, particularly those known as decubitus or decubital ulcers, varicose ulcers, bed sores or pressure sores, in which skin integrity has been breached. Typical treatments for such ulcers include debridement and subsequent wound care. An open ulcer may also be referred to as a “wound”. All or any of these forms, and also burns, may be collectively referred to simply as “ulcer” or “ulcers” herein.

“Burns” is used herein to broadly refer to tissue injury caused by thermal, radiation, chemical, or electrical contact resulting in protein denaturation, burn wound edema, and a loss of intravascular fluid volume due to increased vascular permeability. (See, for example, the Merck Manual, Seventeenth Edition, Ch., 276, pg, 2434-2440.)

“Emollient” refers to a hydrophobic material that has a soothing and moisturizing effect when applied to the skin.

The phrase “vulnerary agents” is used herein to refer to a class of agents, often of plant origin, that are believed to assist in wound healing. Examples include, without limitation, allantoin, various aloe extracts and preparations, balsam [of] Peru, cadexomer iodine, chamomile, chitin, dextranomer, oxaceprol, PDGF (platelet derived growth factor), thioglycerol, and tocoretinoate. The basis of selection is the listing of agents as “vulnerary” in the Merck Index.

“Protease”, when used generically or without qualification, is used herein to refer to a proteolytic enzyme preparation used for debridement. “Debriding enzyme preparation” without qualification refers to enzymes used in debridement, not necessarily limited to proteases.

I. Formulations

Formulations have been developed having several advantages. The formulations are easier and safer to use, longer-lasting, and potentially of higher debridement activity. In some embodiments, the formulation is non-runny and/or non-staining.

A. Emollients

Suitable materials for the emollient include petrolatum, high-melting fatty acids and esters, high-melting triglycerides, lanolin, hydrogenated castor oil, hydroxyethylated castor oil, and hydrogenated hydroxyethylated castor oil, in each case when their melting point is above about 38° C. Additional emollients are well known, and listings can be found can be found in reference books, for example under “Skin Conditioning Agents—Emollient” and “Skin Conditioning Agents-Occlusive” in the “CFTA Cosmetic Ingredient Handbook”, copyright 1988 by the Cosmetics, Toiletries and Fragrance Association of Washington, D.C. Any of the known approved emollients is potentially suitable for use in the composition if it melts above body temperature. Mixtures of emollients can be used. Concentration ranges of between 1 and 15 weight percent are possible with a range of between 2.5 to 5 weight percent preferred. The emollient preferably dissolves or co-emulsifies in the balsam/castor oil/surfactant material when in combination with the propellant, and is selected so that it does not precipitate out or otherwise phase separate when in solution with the propellant at room temperature (approximately 20° C.), or preferably at 15° C. or below. The function of the emollient is to form a firm, non-liquid, non-running protective layer after application of the lotion to the skin or wound, to aid in occlusion. Typically, the higher-melting ranges will be more prone to re-separate from the castor oil and Balsam of Peru oils after the propellant has evaporated.

Another aspect of the present composition is the optional inclusion of a second emollient into the formulation. The second emollient is a hydrophobic material that is a liquid at normal room temperature, for example at about 20° C. The function of the liquid emollient is to improve the cosmetic properties and skin feel of the formulation. Suitable materials for the emollient are well known, and listings can be found in reference books, as noted above. Examples of preferred emollients include vegetable oils, such as safflower oil, olive oil, canola oil, sunflower oil, or avocado oil. Mixtures of liquid emollients can be used. Concentration ranges of from about 1 to about 15 weight percent are possible with a preferred range of from about 2 to about 11 weight percent.

The preferred base ingredient is castor oil. Purified castor oil suitable for pharmaceutical applications, as described in the United States Pharmacopoeia, should be used. Castor oil is typically the largest single component in the formulation, and is implicitly the ingredient required to make up 100% in the formulations described below. Castor oil is preferred because it has been used in approved formulations, and because it is stable indefinitely at room temperature under many conditions. The extra hydroxyl group of the ricinoleic acid may make castor oil more resistant to bacterial and enzymatic attack.

Other USP-grade oils can be used if they are liquid at or near body temperature, preferably at room temperature. Use of such materials requires only simple testing to ensure retention of the enzyme activity. These materials may be conventional vegetable oils, such as corn, canola, peanut, soy, olive and the like; or other plant extracts. Other types of oil include silicone oils and mineral (hydrocarbon) oils. Combinations of oils with each other can also be used.

B. Balsam of Peru

A second key ingredient of the formulation is Balsam of Peru. The purified Balsam of Peru serves both as a part of the vehicle, and as a vulnerary agent in its own right. The Balsam of Peru forms about 1% to about 20% of the liquid composition, preferably about 5% to about 15%, and should be a purified or refined grade. The Balsam of Peru of commerce, unrefined, is a dark colored, viscous, resinous material, with a vanilla-like odor, that has a specific gravity at 25° C. in the range of 1.14 to 1.17. Dilution of unrefined Balsam of Peru with vegetable oil forms a resinous precipitate, which is believed to be the agent causing staining that is found with some prior art products.

Refined Balsam of Peru obtained as the commercially available “refined” material, for example from Polarome International of Jersey City, N.J., is a slightly-viscous liquid described as pale to light brown, having a specific gravity at 250° C. of 1.110 to 1.120, and a refractive index of 1.565 to 1.575.

Purified Balsam of Peru, whether obtained as a commercial refined grade, or purified by the user, can be characterized by having a minimum light transmittance (maximal optical density or absorbance) in the visible and near UV. In particular, suitable material, when diluted with ten volumes of absolute ethanol and measured in a standard 1 cm quartz cell or equivalent versus an ethanol reference, has an optical density at 400 nm wavelength of less than 4.0, preferably less than about 2.0, and more preferably less than about 1.0. The material used in the examples below had an optical density of about 0.4 at 400 nm, on the slope of a steeply rising absorbance peak. (At 380 nm, the optical density was 0.82, and at 375 nm, about 1.0.) In addition, the purified Balsam Peru in ethanol does not immediately form a precipitate, and is not significantly hazy.

C. Enzymes

Debridement agents include proteases, collagenases, and other enzymes that preferentially degrade matrix or other components of necrotic tissues.

In a first preferred embodiment, trypsin is used as the debriding agent, alone or in combination with other debridement agents. Trypsin is a proteolytic enzyme, typically derived from bovine pancreas or alternatively from genetically engineered maize or other organism as a recombinant trypsin. It is believed to debride necrotic tissue by enzymatically hydrolyzing denatured collagen and other extracellular proteins so that the necrotic tissue becomes easier to remove, for example by flushing. The trypsin is suspended as a powder in the formulation. It is stable as a crystalline powder, especially when the pH of a last aqueous solution or suspension was less than about pH 5. In order to obtain a stable crystalline powder of trypsin, crystallization from an acidic solution is preferred. (Stabilization of trypsin is discussed in the art, for example in U.S. Pat. No. 6,177,268). The trypsin is milled to a defined maximum size to avoid plugging the nozzle of the spray device, to provide for a more uniform dispersion of trypsin within the formulation and provide a cosmetically acceptable skin feel. Typically, milling with a 20 mil (500 micron, 0.5 mm) gap, using the formulation as suspending agent, has been found to reduce particle size sufficiently. The particle size is presumed to be less than 500 microns, but the exact size, or its distribution, is not presently known. Alternatively, trypsin powder can be ground and then screened to remove particles above a defined size, for example about 500 microns or about 250 microns, and used as such.

Trypsin is most active at pHs in the range of about 7.0 to 9.0, and can be stabilized by divalent cations (of alkaline earths or transition metals) and/or by polyhydroxyl agents such as glycerol, glycols including polyethylene glycol, sugars (both aldose and alcohol types) and polysaccharides. The amount of trypsin in the formulation may be varied. Concentrations in the range of about 0.005 to about 0.5 weight percent are possible, with the range of about 0.01 to about 0.05 being preferred.

In another aspect, the preferred debridement enzyme is collagenase, an enzyme that is specific for collagen as a substrate and lyses different peptide bonds than does trypsin. Collagenase can also be delivered in a finely divided solid form to the damaged tissue. Concentrations in the range of about 0.005 to about 0.5 weight percent are possible, with the range of about 0.01 to about 0.05 weight percent being preferred.

Additional debridement agents may be combined into the formulation. These may include papain and/or other vegetable-derived proteases.

D. Additional Actives/Local Anesthetics

The process of cleaning an ulcer is painful to the patient. It is advantageous, and may increase compliance, if a topical anesthetic spray is packaged with the debridement formulations. It may be applied at the beginning of the cleaning procedure, and optionally again just before application of the formulation.

During the process of wound healing, itching can become a serious problem. Scratching the healing wound destroys the newly granulated tissue, delaying the healing process. The increased emolliency imparted by the use of either emollients which are solid at room temperature or thickeners which increase the viscosity of the formulations can help to reduce the itching that accompanies the regranulation of wounds. The reduction in itching reduces the incidence of scratching and thereby prevents the destruction of newly granulated tissue, thereby helping to promote efficient tissue growth and wound healing.

The formulations may contain additional medicaments that promote wound healing, prevent contamination of the treatment site by bacteria, i.e. bacterial infection, reduce and/or eliminate the inflammation caused by bacteria, reduce and/or eliminate the unpleasant sensations associated with burns, pressure sores, dermal ulcers, varicose and decubital ulcers. The examples of such medicaments include but are not limited to various antibiotics and locally and centrally acting pain killers.

E. Enhancers of Enzyme Activity

The formulation is designed to create conditions for optimal debridement enzyme action as part of the application of the product. The function of the enzyme is to assist in tissue debridement. The enzyme powder is typically stable in the anhydrous formulation, and dissolves and becomes active when it encounters fluid emanating from the tissue surrounding the ulcerated site, for example as serum. Many enzymes, including trypsin and collagenase, are most active at slightly alkaline pH; and whatever the pH optimum of the particular enzyme, activity will be greater or more prolonged if the pH is maintained at that value.

The debridement enzyme preparation can be supplemented with one or more enhancers for obtaining maximal activity and/or duration of activity of the enzyme once the formulation is applied onto the treatment site. Enhancers include divalent salts, particularly calcium salts, or other salts required for enzymatic activity. Enhancers may include buffers, to maintain the pH in the range optimal for the specific enzyme used, for example pH 6 to pH 9, preferably pH 7 to pH 8 for trypsin. Enhancers may include stabilizers, particularly polyol stabilizers, including glycols, glycerol, erythritol, sorbitol, inositol, pentoses, hexoses, oligosacchaides and polysaccharides. Glycols include polyethylene glycol and other polyalkylene glycols. Combinations of such enhancers are preferred. The enhancers may be mixed with a powdered or other form of the enzyme, or may be provided in a separate container. The enhancers may also or instead be provided separately from the enzyme preparation. Enhancers, when not liquid, are preferably provided as finely divided powders that will dissolve readily in wound exudates.

Typically, enhanced enzyme activity and/or stability is found when certain co-factors are present. These are largely simple materials, often materials found intracellularly, or sometimes in the blood or other extracellular fluid, that activate the particular enzyme. For example, trypsin prefers the presence of millimolar quantities of alkaline earth cations such as Ca++ or Mg++ (as does chymotrypsin and subtilisin), while collagenase prefers transition metal ions such as Zn++. Bromelain, papain and chymopapain prefer an environment with reduced sulfhydryl groups (RSH). Each debriding enzyme will require a particular set of cofactors, and for known enzymes, the cofactors are generally known to persons skilled in biochemistry and usually can be readily obtained.

Besides salts, enhancers may include buffers, to maintain the pH in a particular range preferred by the enzyme, for example, for trypsin or collagenase, pH 6 to pH 9, preferably pH 7 to pH 8. Enhancers may also include stabilizers, particularly polyol stabilizers, including glycols, glycerol erythritol, sorbitol, inositol, pentoses, hexoses, oligosacchaides and polysaccharides. Glycols include polyethylene glycol. Combinations of such enhancers are preferred. The enhancers may be mixed with a powdered or other form of the enzyme, or may be provided in a separate container. The enhancers may be provided separately from the enzyme preparation. Enhancers, when not liquid, are preferably provided as finely divided powders that will dissolve readily in wound exudates.

Trypsin is most stable in the presence of calcium or magnesium ions. While some magnesium is present in serum, neither the divalent ion concentration nor the pH of exuded fluid at the ulcerated site is predictable. The formulation is improved to remove this deficiency by the inclusion of materials that will supply buffering action and/or divalent metal salts at the site of administration. Any of a variety of buffers and divalent ions can be used. Some compatible buffers and stabilizing ions are named in U.S. Pat. No. 6,177,268, and other buffers can be found in chemical and biochemical catalogs.

In one embodiment, micronized buffer salts and/or magnesium or calcium salts are added to the formulation, along with the trypsin. Upon hydration, the micronized salts and/or buffers dissolve in bodily fluids, such as exuded serum, thereby adjusting the pH of the exudate, and providing divalent ions for stabilization, resulting in the maintenance of full trypsin activity over a prolonged period of time. Examples of buffers include but are not limited to the sodium salts of phosphoric acid, N-(2-hydroxyethyl)piperazine-N′-(2-ethanesulfonic acid), 1,4-piperazinediethanesulfonic acid, N,N-bis(2-hydroxyethyl)-2-aminoethanesulfonic acid and 3-(N-morpholino)propanesulfonic acid. Examples of soluble magnesium and calcium salts include but are not limited to magnesium chloride, magnesium acetate, magnesium citrate, magnesium lactate, calcium acetate, calcium lactate, calcium citrate and calcium gluconate. Polyhydroxy materials, including but not limited to sugars, glycols, glycerol PEG, and polysaccharides, can also be provided to stabilize trypsin once it is on the tissue surface. The polyols can be dissolved in the formulation, or present as fine powders, below 500 microns in diameter, as in the case of the trypsin and buffer salts.

Urea is a known moisturizer and is used in skin care products to enhance skin water retention. Urea may be added to the formulations to enhance water retention at the site of treatment, thus providing a favorable environment for the enzymatic debridement. Urea may also soften the skin making it more accessible to the enzymes. Urea is used in the concentration that does not denature the enzyme being used. Such concentration can be experimentally determined for each individual formulation. Some enzymes, such as papain, are tolerant of moderate levels of urea, and its effect on other proteins may make them easier for the papain to digest.

Because the amount of aqueous solution in the wound, in which the buffer and the enzyme will initially dissolve upon administration of the formulation, is not fixed, the resultant composition on the skin surface will necessarily be inexact. Ranges of buffer salts of about 0 to 5 weight percent, and of divalent ion salts of about 0.1 to about 5 weight percent are expected to provide reasonable ion concentrations and pHs in exuded fluid at various volumes.

The buffer and the divalent ions may be formulated as a single powder if desired. Powderable polyol stabilizers can optionally be included in the powder. As with the enzyme powder, powdered excipients can be prescreened to be of a suitable size for use in the spray apparatus provided, or can be ground to an appropriate size in a liquid component of the formulation.

F. Emulsifiers, Emollients, Surfactants, Viscosity Modifiers

An emulsifier is desirable to help promote efficient release of the actives from the formulation and to improve the ease of clean-up of the wound site. A wide variety of surfactants are potentially useful. Useful nonionic surfactants include Oleth-10 (polyoxyethylene (10) oleyl ether) in a range from about 1% to about 15%, and presently preferred at about 2% to 7%. Those skilled in the art would be able to test other surfactants, beginning with those having similar HLB, in order to arrive to stable formulations. Mixtures of surfactants can be used to optimize the properties of the formulation.

Any pharmaceutically or cosmetically acceptable thickener suitable for thickening hydrocarbon, silicone or vegetable oils may be used in the formulations. The thickeners modify the rheology of the formulations in order to establish the proper balance between activity and, application and post application physical behavior. Examples of thickeners include colloidal silicas and starches. An example of a preferred thickener is colloidal silica. The thickener is used in a concentration range of between 1.0% to about 5.0%, more preferably in a range of between 1.0% and 2.5%. Those skilled in the art would be able to test other thickeners in order to prepare stable formulations. Mixtures of thickeners can be used to optimize the properties of the formulation.

In a preferred embodiment, the formulation can be efficiently delivered from a dispensing device such as a pump dispenser or an aerosol can, and has a sufficiently high viscosity to prevent the formulation from “running off” the site of application. The formulation also preferably has a sufficiently low viscosity that the formulation efficiently releases the actives, particularly the debriding enzyme(s), to the wound site. These contrasting attributes can be realized by using a shear-thinning formulation. “Shear-thinning” describes the rheological condition where the viscosity of a material subjected to constant shear stress decreases. The amount of decrease in viscosity is a function of the degree of shear stress applied. Upon removal of the shear stress, the viscosity again increases to the original value over time. Two values are of importance in developing shear-thinning formulations for wound care. These are the zero shear viscosity and the critical stress. The zero shear viscosity dictates the resistance of the formulation to flow after application and ability of the formulation to release actives. The critical stress defines the stress level at which the material transitions from a “solid-like” poorly flowing high viscosity fluid to a “liquid-like” well flowing low viscosity fluid.

The lower the critical stress is, the easier it is for the formulation to be dispensed from the packaging, and the lower the force needed to uniformly spread the formulation on the wound site. For topical wound care products, the preferred zero shear viscosities are preferably greater than about 15,000 cP, more preferably greater than about 25,000 cP, and most preferably greater than about 35,000 cP; and also less than about 700,000 cP, more preferably less than about 550,000 cP and still more preferably less than about 450,000 cP when measured at room temperature. The preferred critical stress values are preferably greater than about 25 dynes/cm², more preferably greater than about 75 dynes/cm², and most preferably greater than about 100 dynes/cm² , and also preferably lower than about 4,000 dynes/cm², more preferably lower than about 3,000 dynes/cm², still more preferably lower than about 2,500 dynes/cm², and still more preferably less than 1,500 dynes/cm².

Urea and other chaotropic agents are known moisturizers and are used in the skin care products to enhance skin water retention. Urea may be added to the formulations to enhance water retention at the site of treatment thus providing favorable environment for the enzymatic debridement. Urea may also soften the skin making it more accessible to the enzymes. Urea is used in the concentration that does not denature the enzymes. The concentration can be experimentally determined for each individual formulation.

The formulation may contain any of a variety of conventional additives and excipients. These can include, without limitation, viscosifiers, additional occluding agents, fragrances, deodorants, colorants, preservatives, vitamins and other skin nutrients, antioxidants, and other stabilizing agents. The various components described above can be collected and provided as a kit.

G. Propellants/Aerosol or Dry Powder Formulations

Sprays

The formulations can be dispensed by spraying. In one embodiment, the enzyme preparations are sprayed as a dry powder from a pressurized can, or less preferably from a hand-pumped container. In a pressurized can, any medically approved propellant is potentially suitable, including alkanes such as propane and butane, and approved hydrofluoroalkanes, such as tetrafluoroethane (HFA 134a) and heptafluoropropane (HFA 227). Optionally and preferably, the preparation to be sprayed contains enhancers. The formulation may contain a surfactant to maintain the various ingredients in a single phase, or as a two-phase preparation that will re-emulsify upon brief shaking.

The spray may contain other sprayable components. These may include oily or occlusive materials, such as vegetable oil, or a polymer that is soluble in the propellant but that precipitates on the skin as the solvent evaporates. The spray solution in the can may also contain surfactants, to keep the components mixed. It may also contain combinations of surfactants and polymers that will foam on emergence from the aerosol can. The foam will carry the debridement enzymes, such as trypsin, or collagenase, and optionally enhancers, and will deposit these active ingredients on the skin in a non-running, well-localized manner. The foam will preferably collapse, immediately or gradually, and preferably upon contact with tissue exudates, thereby delivering the enzymes to the tissue surface.

Any pharmaceutically acceptable hydrocarbon, CFC or HFA propellant can be used in the formulations. The preferred propellant of an aerosol formulation is a HFA (hydrofluoroalkane, also known as hydrofluorocarbon, HFC), such as HFA 134a (tetrafluoroethane) or HFA 227 (heptafluoropropane) or other HFA approved for medical use. The HFAs have a much lower ozone destroying potential than chlorofluorocarbons (CFCs) and are currently approved as propellants. They are non-flammable, unlike the alkane propellants, such as propane and butane. In the literature, HFAs are often used with irritating and/or flammable co-solvent materials, such as ethanol and other lower alcohols, to reduce pressure. A co-solvent is not necessary in the formulations. The HFA is charged to the spray container so as to form about 10% to about 50% of the final weight of the container's contents, more preferably at about 15% to about 40%, still more preferably at about 20% to about 35%. The emollient preferably dissolves or co-emulsifies in the balsam/castor oil/surfactant material when in combination with the propellant, and does not precipitate out or otherwise phase separate when with the propellant at room temperature (ca. 20° C.), or preferably at 15° C. or below.

The spray can is conventional, and preferably is aluminum with an inner coating of epoxy or other passivating lining. A preferred feature of the spray can is a multi-angle spray head/dispenser, which can dispense the formulation from angles other than purely upright.

In an alternative embodiment, one can provide a kit containing the enzyme-containing concentrate formulation, in aerosol or lotion form, and a sterile aqueous rinse solution in a hand pumped spray bottle or other simple container, containing physiological levels of divalent ions, and buffered to an appropriate pH, for example in the range of 7 to 9, or for example about pH 8. Molarities of buffer can be in the range of about 5 to 200 mM; of divalent ions in the range of about 1 to 50 mM. The last stage of cleaning the ulcer is followed by a rinse with the buffered alkaline solution, which is followed by the application of the formulation containing enzyme from its dispenser. Protective polyols can be included either in the physiologic solution, or in the formulation with the enzyme. Amounts of polyols are not rigidly fixed; 0.1 to 5% by weight in an aqueous solution or 0.1 to 10% in the concentrate would provide at least some improvement in stabilization of the enzyme.

In another embodiment, prior to incorporation into the formulation, an intimate mixture of enzyme and activators/stabilizers is formed, for example, by co-dissolving all desired ingredients in an aqueous medium and then freeze-drying or spray-drying the resultant solution. In this embodiment the addition of activators and/or stabilizers to the formulation as a separate component is optional.

Dry Powders

In another aspect, the enzyme can be delivered in a solid form to the damaged tissue. In one embodiment, the enzyme is deposited on a substrate, and dissolves on contact with tissue exudates, or in activating fluid. For example, an enzyme solution can be spray-dried onto a mesh or gauze, or into a porous material such as an open celled sponge or membrane. Preferably, this is done in an aseptic dispensing mode onto pre-sterilized substrate. The substrate is applied to the area to be treated, and optionally the carrier can be trimmed to allow the shape of the dispensing carrier to be the same as that of the wound. This will minimize the effect of the enzyme on undamaged tissue. A patch containing dry enzyme can be provided.

The enzyme either dissolves on contact with tissue exudates, or in an activating solution that is sprayed on the treatment site or onto the enzyme-containing side of the patch. The patch may contain enhancers described above. One or more enhancers may be added to the activating solution. The enzyme can be activated by exudates, or an applied hydrating solution, or a combination. The applied solution can contain the enhancers described above, and optionally is dry and is reconstituted at the time of use with sterile water. A simple hand sprayer can be used to hydrate the enzyme and wash it into the wound. Alternatively, a sponge full of water or hydrating solution can be applied. An occlusive dressing can be applied to retain the active enzyme in the wound site for the duration of the treatment. Optionally, materials can be present in the hydrating solution, or in dry form on the carrier, that will make the enzyme solution viscous or gelled, to help maintain its contact with the site to be treated.

Optionally, a unit-of-use package containing a dry enzyme material to be reconstituted in an aqueous solution with or without enhancers prior to use may be provided. The obtained enzyme solution can be applied onto the skin using a pipette, spray bottle, and/or brush.

The present invention will be further understood by reference to the following non-limiting examples.

### EXAMPLE 1

### Trypsin Formulations Including Castor Oil and Balsam of Peru

To make the formulations, castor oil is placed in a stirred container and heated to 85° C. Hydrogenated castor oil is added to the castor oil and the mixture is stirred until the hydrogenated castor oil is fully dissolved. The resulting mixture is cooled while stirring to 40° C. Balsam of Peru oil and polyoxy 10 oleyl ether are then added and the mixture is stirred until uniform. In a mortar and pestle, safflower oil and trypsin are ground together to form a smooth mixture. The trypsin/oil mixture is then added to the castor oil mixture and stirred until uniform and smooth. After the mixture returns to room temperature, the finished formulation is packaged in standard pump dispensing packaging. This procedure was used to prepare the formulations given in Table 1 below.

TABLE 1
Formulations

| | Formulation 1 | Formulation 2 | Formulation 3 |
|---|---|---|---|
| Ingredient | Weight % | Weight % | Weight % |
| Castor Oil | 76.3 | 73.8 | 75.05 |
| Hydrogenated | 2.5 | 5 | 3.75 |
| Castor Oil | | | |
| Balsam of Peru Oil | 8.7 | 8.7 | 8.7 |
| Polyoxy 10 | 2 | 2 | 2 |
| Oleyl Ether | | | |
| Safflower Oil | 10.488 | 10.488 | 10.488 |
| Trypsin | 0.012 | 0.012 | 0.012 |

### EXAMPLE 2

### Trypsin Formulations also Containing Colloidal Silica

To make the formulations, castor oil, safflower oil, balsam peru oil and polyoxy 10 oleyl ether are combined and stirred under low shear conditions until a uniform solution is obtained. Trypsin is then dispersed uniformly into the solution by low shear mixing. The colloidal silica is then dispersed into the suspension by low shear mixing to form a uniform suspension. The slurry is then subjected to brief high shear mixing to fully disperse the trypsin and colloidal silica allowing the suspension viscosity to fully develop. The finished formulation is packaged in standard pump dispensing packaging. This procedure was used to prepare the formulations given in table 2 below.

TABLE 2
Formulation

| | Formu- | Formu- | Formu- | Formu- | Formu- |
|---|---|---|---|---|---|
| | lation | lation | lation | lation | lation |
| | 4 | 5 | 6 | 7 | 8 |
| Ingredient | Weight % | Weight % | Weight % | Weight % | Weight % |
| Castor Oil | 78.8 | 78.8 | 78.8 | 78.8 | 78.8 |
| Colloidal | 2.5 | 1.75 | 3.5 | 2.5 | 2.0 |
| Silica | | | | | |
| Balsam of | 8.7 | 8.7 | 8.7 | 8.7 | 8.7 |
| Peru Oil | | | | | |
| Polyoxy 10 | 2 | 2 | 2 | 2 | 2.0 |
| Oleyl Ether | | | | | |
| Safflower | 7.988 | 8.738 | 6.988 | 4.588 | 5.088 |
| Oil | | | | | |
| Trypsin | 0.012 | 0.012 | 0.012 | 0.012 | 0.012 |
| Calcium | 0.0 | 0.0 | 0.0 | 1.04 | 1.04 |
| Lactate | | | | | |
| HEPES | 0.0 | 0.0 | 0.0 | 2.36 | 2.36 |

### EXAMPLE 3

### Aerosol Trypsin Formulation 9

To make a Formulation concentrate, castor oil (738 gm) is placed in a stirred container. In separate containers, balsam oil (93 grams) and Oleth-10 surfactant (68 gm) are heated to 50° C., and the heated ingredients are added to the castor oil. The mixture is stirred to blend it. A portion of the mixture (about 100 gm) is taken and 1 gm of trypsin is added to it. The trypsin/oil mixture is processed in a colloid mill for at least 5 passes. Meanwhile, 50 g of polyoxyl 60 hydrogenated castor oil, m.p. ca. 40° C., is melted and added with stirring to the castor oil mixture. After the mixture returns to room temperature, the trypsin/castor oil dispersion is added with stirring. The finished Formulation concentrate is charged to aluminum spray cans (90 g), and then 28 g per can of HFC 134a is added under pressure. A satisfactory spray is obtained, resulting in a thick, viscous coating that becomes waxy as the propellant evaporates.

### EXAMPLE 4

### Aerosol Trypsin Formulation 10 Containing Colloidal Silica

To make a Formulation concentrate, castor oil (788 gm) is placed in a stirred container. Balsam oil (87 grams), Oleth-10 surfactant (20 gm) and Safflower Oil (87.38 gm) are added to the castor oil. The mixture is stirred to blend it. A portion of the mixture (about 100 gm) is taken and 0.12 gm of trypsin is added to it. The trypsin/oil mixture is processed in a colloid mill for at least 5 passes. The trypsin/castor oil dispersion is then added back to the bulk of the Formulation with stirring. 17.5 gm Colloidal Silica is then added to the mixture and blended to disperse. The Formulation concentrate is then mixed with a high shear mixer to fully develop the final viscosity. The finished Formulation concentrate is charged to aluminum spray cans (90 g), and then 28 g per can of HFC 134a is added under pressure. A satisfactory spray is obtained, resulting in a viscous coating that develops as the propellant evaporates.

### EXAMPLE 5

### Activity Testing of Formulations 4-8

The enzymatic activity of Formulations 4-8 was measured by their ability to generate a zone of clearance on casein-agar plates. This test mimics in vivo scenarios wherein a topical Formulation is applied on the treatment site. Formulation 1 and 2 were tested along with a commercial trypsin ointment. The experimental procedure was as follows:

Prepare 50 mls of Casein-Agar

Transfer 5 mls agar to 60 mm petri dish, repeat 9 times

Allow to solidify at RT (room temperature)

Warm test Formulations to 40° C.

Apply 100 microliters of drug product to the surface of the agar and spread to cover a defined area.

Incubate at 40° C.

Measure zone of clearance at 1 hour, 2 hours, 4 hours and 6 hours

The casein-agar plates are opaque white in appearance. As the trypsin digests the casein the plates become clear. Clearance was rated from 0 to 5 using the following scale:

| Rating | Definition |
|---|---|
| 0 | no clearing visible in treated area |
| 1 | outline of treated area evident |
| 2 | clearing easily visible in treated area |
| 3 | treated area 50% cleared |
| 4 | treated area 75% cleared |
| 5 | treated area completely cleared |

Each time point was measured in triplicate and the experiment was repeated three times. The results are the average of all measurements for each time point. The viscosity of the tested Formulations varied, with the commercial trypsin ointment being the most viscous and Formulation 1 being the least.

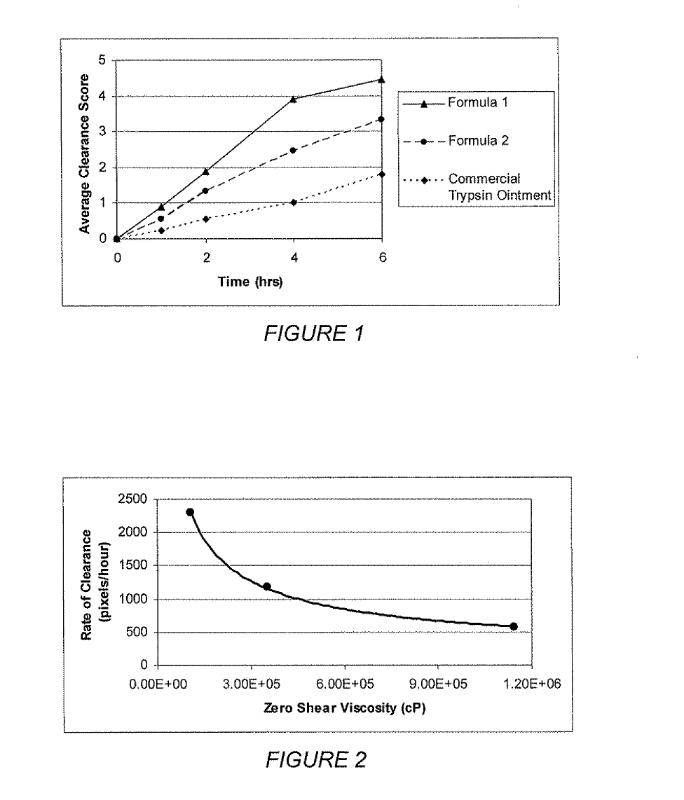

FIG. 1 demonstrates that the relative activities of the Formulations at any given time point are superior to the prior art material. It is possible, especially between Formulation 1 and Formulation 2, that the difference in apparent enzyme activity corresponds to the test article's relative viscosity, so that as the viscosity of the Formulation increases the observed activity of the Formulation decreases.

Therapeutically suitable non-aqueous lotion Formulations should have sufficiently high viscosity that they do not run off the site of application and provide some itch relief. In addition, the data above indicate that a sufficiently low viscosity in the Formulation is important for maximal enzyme activity. The enzymatic activity of Formulations 4-6 were determined and plotted against their zero shear viscosities. The experiment was carried out as described below:

Prepare 90 mls of Casein-Agar

Transfer 5 mls agar to 60 mm petri dish, repeat 17 times

Allow to solidify at RT

Apply 100 microliters test Formulation to 13 mm diameter nitrocellulose membrane filters

Apply 3 treated filters to each plate

Incubate at 40°

Measure zone of clearance at 1 hour, 2 hours and 3 hours

Image the plates at each time point with a digital camera

Quantitate the area of clearance using Scion Image

The plates were analyzed as described above. The zero shear viscosity for each Formulation was determined by linear regression of shear stress versus viscosity plots. Each activity time point was measured in duplicate and the experiment was repeated three times. The results are the average of all activity measurements for each time point. The activity data was plotted as a function of time and the rate of clearance was determined from the slope of the regression line. The table and graph below lists the rate of clearance and the zero shear viscosity of each of the tested Formulations.

TABLE 3
Efficacy of Trypsin Formulations

| Formulation | Rate of Clearance | Zero Shear Viscosity |
|---|---|---|
| 4 | 1180 pixels/hour | 353000 cP |
| 5 | 2298 pixels/hour | 105000 cP |
| 6 | 574 pixels/hour | 1143000 cP |

Table 3 and FIG. 2 show the rate of clearance and zero shear viscosity for Formulations 4-6. The results of the experiment demonstrate that the activity of the trypsin against a solid substrate directly correlates with the viscosity of the Formulation.

Soluble divalent metal salts are known to stabilize the activity of trypsin in solution. Buffers control the pH of an aqueous solution and, hence, influence trypsin activity in it. It is not clear however if this behavior would allow the stabilization of activity against a solid substrate. To demonstrate that it is possible to stabilize the activity of trypsin against solid substrates Formulations 7 and 8 were tested using the protocol described below:

Prepare 60 mls of Casein-Agar

Transfer 5 mls agar to 60 mm petri dish, repeat 11 times

Allow to solidify at RT

Apply 100 microliters test Formulation to 13 mm diameter nitrocellulose membrane filters

Apply 3 treated filters to each plate

Incubate at 40

Measure zone of clearance at 1 hour, 2 hours and 3 hours

Image the plates at each time point with a digital camera

Quantitate the area of clearance using Scion Image

Each time point was measured in duplicate and the experiment was repeated twice. The results are the average of all measurements for each time point.

TABLE 4
Efficacy of Trypsin Formulations

| Formulation | Rate of Clearance | Zero Shear Viscosity |
|---|---|---|
| 7 | 11426 pixels/hour | 358000 cP |
| 8 | 6909 pixels/hour | 855600 cP |

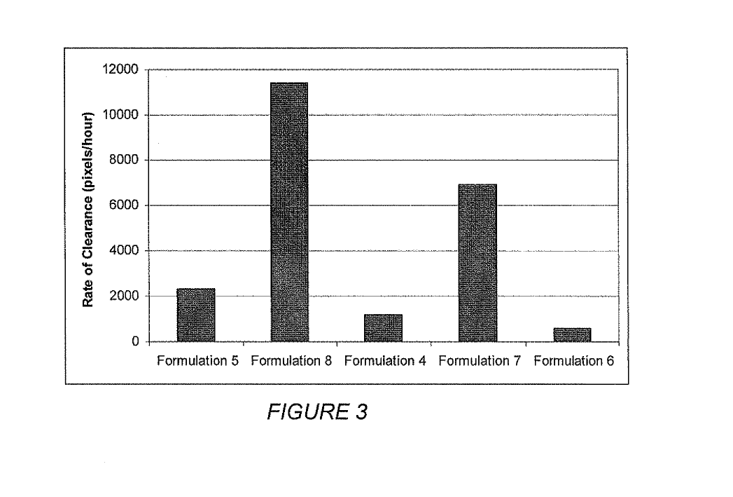

FIG. 3 and Table 4 show the results of the experiment. The observed difference in activity between Formulations containing trypsin and those containing trypsin with activity stabilizers demonstrates the ability of the calcium/buffer combination to stabilize the activity of trypsin against a solid substrate.

The examples and descriptions of the specification are intended to illustrate the invention and to aid in understanding it, and will suggest other embodiments within the scope of the invention to the skilled person. The invention is not limited in scope by the specific description given, but only by the claims.

We claim:

1. A topical composition for the treatment of open wounds, burns and ulcerated skin conditions, the composition comprising:
a lipophilic base selected from the group of vegetable oils, silicone fluids, mineral oils, fatty acids, glycerides and combinations thereof;
a surfactant comprising between about 1% to about 15% of the weight of the composition;
a medically effective amount of one or more topically active vulnerary agents;
a medically effective amount of a debriding enzyme preparation; and
one or more emollients, comprising from about 0,5% to about 30% of the weight of the composition,
wherein the composition has a yield stress between 25 and 4,000 dynes/cm².

2. The composition of claim 1 further comprising an amount of a pharmaceutically acceptable thickening agent comprising between 1% and 5% of the composition.

3. The composition of claim 1 where at least one of the emollients melts at about 38° C. or above.

4. The composition of claim 1 where at least one of the emollients is a liquid at room temperature.

5. The composition of claim 1, comprising a first emollient which melts at or above 38° C., and one or more second emollients which are liquid at room temperature.

6. The composition of claim 1 comprising an emollient compound having a melting point at or above about 38° C. selected from the group consisting of petrolatum, high-melting fatty acids and esters, high-melting triglycerides, lanolin, hydrogenated castor oil, hydroxyethylated castor oil, and hydrogenated hydroxyethylated castor oil.

7. The composition of claim 2 where the pharmaceutically acceptable thickening agents are selected from a group consisting of colloidal silicas, starches, clays and metal oxides.

8. The composition of claim 1 where the vulnerary agents are selected from the group consisting of Balsam of Peru, Povidone-Iodine, Cadexomer-Iodine, and Silver Nitrate.

9. The composition of claim 1 comprising a purified Balsam of Peru oil, the Balsam of Peru Oil having a specific gravity at 25° C. of 1.110 to 1.120, and a refractive index of 1.565 to 1.575.

10. The composition of claim 1, comprising a purified Balsam of Peru oil, the Balsam of Peru Oil having an optical density, after dilution with ten volumes of absolute ethanol, of less than about 4 in a 1 cm quartz cell at 400 nm.

11. The composition of claim 1 where the debriding enzyme is selected from the group consisting of trypsin, chymotrypsin, papain, chymopapain, subtilisn, bromelain and collagenase.

12. The composition of claim 1 wherein the enzyme is in the form of a powder having a number average particle size of less than about 500 microns, preferably less than about 150 microns, more preferably less than about 50 microns.

13. The composition of claim 1 wherein the yield stress of the Formulation is between 25 and 1,500 dynes/cm².

14. The composition of claim 1 where the zero shear viscosity of the Formulation is between 15,000 and 700,000 cP.

15. The composition of claim 1 further comprising a suspension of finely powdered dry buffer, wherein the buffer will produce a pH in the range of about 7 to about 9 when in contact with an aqueous exudate from the flesh of the patient.

16. The composition of claim 1 further comprising a suspension of finely powdered dry buffer, wherein the buffer will product a pH in the range of about 5 to about 7 when in contact with an aqueous exudates from the flesh of the patient.

17. The composition of claim 1 further comprising a suspension of one or more finely powdered water soluble salts of divalent metals.

18. The composition of claim 1 further comprising a suspension of finely powdered mild reducing agent.

19. The composition of claim 18 comprising a suspension of finely powdered urea.

20. The composition of claim 1 further comprising polyols dissolved or suspended in the composition.

21. The composition of claim 1 comprising one or more powders comprising buffer, divalent metal salts, polyols, or a combination thereof, having a diameter of less than about 500 microns, preferably less than about 150 microns, more preferably less than about 50 microns.

22. The composition of claim 1 in admixture with a pharmaceutically acceptable propellant comprising from about 10% to 50% of the weight of the final composition.

23. The composition of claim 1 in admixture with a pharmaceutically acceptable propellant comprising from about 15% to about 40% of the final composition.

24. The composition of claim 1 comprising a medicinally-acceptable hydrofluoroalkane (HFA) propellant the HFA comprising from about 1.0% to 50% of the weight of the final composition.

25. The composition of claim 1 in a first container in a kit for the treatment of ulcers, the kit further comprising a second container containing a topical anesthetic.

26. The composition of claim 1 in a first container in a kit for the treatment of ulcers, the kit further comprising a second container containing an aqueous solution, the solution comprising a buffer adjusted to a pH between about pH 7 and about pH 9; at least about 1 mM of divalent metal salts; at least about 1% of polyols; and the solution being approximately isotonic in osmolarity.

27. The composition of claim 1 in a patch to be applied to the wound site.

28. The composition of claim 1 in a spray can.

29. The composition of claim 28 in a kit further comprising a solution in a second sprayable container.

30. The composition of claim 29 the second spray can or second container can spray in any orientation.

31. A dry powder composition for the treatment of open wounds, burns and ulcerated skin conditions, comprising a medically effective amount of a debriding enzyme preparation applied to a sterile substrate appropriate for application to an open wound,

32. The composition of claim 31 further comprising a finely powdered dry buffer.

33. The composition of claim 31 further comprising one or more finely powdered water soluble salts of divalent metals.

34. The composition of claim 31 further comprising finely powdered urea.

35. The composition of claim 31 further comprising finely powdered cysteine.
